(19) United States
(12) Patent Application Publication
(10) Pub. No.: US 2007/0065851 A1
(43) Pub. Date: Mar. 22, 2007

(54) Method for detecting a microorganism in a fecal specimen

(75) Inventor: Arie Anton van Zwet, Wapenveld (NL)

Correspondence Address: TRASK BRITT, P.O. BOX 2550, SALT LAKE CITY, UT, 84110, US

(73) Assignee: Stichting Laboratorium voor Infectieziekten, Groningen (NL)

(21) Appl. No.: 11/510,287

(22) Filed: Aug. 25, 2006

Related U.S. Application Data
(63) Continuation of application No. PCT/NL05/00138, filed on Feb. 25, 2005.

(30) Foreign Application Priority Data
Feb. 27, 2004 (EP) 04075632.2

(51) Int. Cl. C12Q 1/68; C07H 21/04

(52) U.S. Cl. 435/6; 536/23.7

(57) ABSTRACT

The invention relates to detecting microorganisms in specimens for the purposes of inter alia diagnoses, screenings, quarantine inspections, and clinical tests. Specifically, it relates to detecting and quantifying enteropathogenic bacteria in a fecal specimen, including Shigella species, Salmonella species, Campylobacter species, enterohemorrhagic E. coli or Verocytotoxin-producing E. coli, Vibrio cholerae, and Clostridium perfringens. Provided is a method for detecting a microorganism in a fecal specimen, wherein the method comprises preparing a 25-50% (wt/vol) fecal lysate, optionally followed by isolating a nucleic acid from the fecal lysate; subjecting the nucleic acid to a nucleic acid amplification assay using a set of at least two nucleic acid amplification primers specific for the microorganism and detecting amplified nucleic acid to determine the presence of the microorganism in the specimen.

### CROSS-REFERENCE TO RELATED APPLICATION

This application is a continuation of PCT International Patent Application No. PCT/NL2005/000138, filed on Feb. 25, 2005, designating the United States of America, and published, in English, as PCT International Publication No. WO 2005/083122 on Sep. 9, 2005, the contents of the entirety of which is incorporated by this reference, which application itself claims priority to EP 04075632.2 filed Feb. 24, 2004.

### BACKGROUND

The present invention relates to detection of microorganisms in specimens for the purposes of inter alia diagnoses, screenings, quarantine inspections, and clinical tests. Specifically, it relates to detection and quantification of enteropathogenic bacteria in a fecal specimen, including Shigella species, Salmonella species, enterohemorrhagic Escherichia coli or Verocytotoxin-producing Escherichia coli, Vibrio cholerae, Campylobacter species and Clostridium perfringens.

Detection of pathogenic bacteria such as Shigella species, Salmonella species, enterohemorrhagic Escherichia coli (hereinafter referred to as EHEC) or Verocytotoxin-producing Escherichia coli (hereinafter referred to as VTEC), Vibrio cholerae, and Clostridium perfringens is an important task in the field of medicine and public hygiene, and various methods have been used. Conventionally, detection of a pathogenic bacterial strain involves isolation of several pathogenic bacterial colonies and identification of the species of the bacteria by serological or biochemical methods.

In the case of Shigella species, this has been achieved by culturing and isolating the target bacterium from specimens of patient stools, food, or the like, using a medium, such as DHL agar or MacConkey agar, and then further culturing the bacterium using a medium such as TSI agar or LIM agar for the purpose of identification.

In the case of Salmonella species, culturing is conducted for isolation of the bacteria from specimens of patient stools or vomits, food or wiping samples, etc., followed by inoculation to TSI agar, SIM medium, VP-MR medium or lysine decarboxylation test medium and subsequent overnight culture at 37° C., to confirm Salmonella species, and the serotype is determined using a commercially available set of antisera against O and H antigens.

EHEC or VTEC has been found to cause hemolytic uremic syndrome in children, as well as food poisoning symptoms, typically hemorrhagic colitis, and recently stress has been placed on detection of this bacterium in clinical tests. In the case of detecting EHEC or VTEC, specimens are patient stools, food, or water samples (drinking water, river water, etc.) collected from the environment surrounding the patient. In detecting EHEC (VTEC) in these specimens, it is necessary to perform a series of procedures comprising direct isolation culture, a primary confirmation culture test, and a secondary confirmation culture test and agglutination test with an antiserum.

In the case of Vibrio cholerae, specimens are patient stools or food, water samples (drinking water, river water, sea water, etc.) or samples collected from the environment surrounding the patient. In detecting and identifying Vibrio cholerae in these specimens, it is necessary to perform a series of procedures from primary enrichment culture, secondary enrichment culture, and isolation culture to an agglutination reaction test with specific anti-V cholerae O group 1 antiserum and confirmation culture.

In the case of Clostridium perfringens, specimens are obtained mainly from patient stools and food. For detection and identification, the specimens are subjected to enrichment culture and isolation culture under anaerobic conditions. With several colonies of the bacteria, tests for biochemical properties are conducted.

Nucleic acid amplification technology, such as polymerase chain reaction-(PCR) based assays, allow detection of a microorganism without having to grow the microorganism in culture. Therefore, PCR-based methods are much faster compared to DNA probing or hybridization techniques. The detection of microorganisms in a biological sample by PCR can generally be divided into four steps: (1) sample collection, (2) sample preparation, (3) nucleic acid (DNA) amplification by PCR and (4) detection of amplified PCR products.

PCR has become a very rapid and reliable tool for the molecular biology-based diagnosis of a variety of micro-organisms. PCR has been applied for the detection of microorganisms from microbial cultures and directly from clinical samples. However, a major problem with PCR is that it is very sensitive for inhibition. Fecal specimens are among the most complex specimens for direct PCR testing (i.e., without culturing the specimen to enrich for micro organisms of interest) due to the presence of inherent PCR inhibitors that are often co-extracted along with the microbial DNA (see for example Brian et al., 1992 “Polymerase chain reaction for diagnosis of enterohemorrhagic Escherichia coli infection and haemolytic-uremic syndrome” J. Clin. Microbiol. 30:1801-1806; Stacy-Phipps et al., 1995 “Multiplex PCR assay and simple preparation method for stool specimens detect enterotoxigenic Escherichia coli DNA during course of infection” J. Clin. Microbiol. 33: 1054-1059; Wilde et al., 1990 “Removal of inhibitory substances from human fecal specimens for detection of group A rotavirus by reverse transcriptase and polymerase chain reactions” J. Clin. Microbiol. 28:1300-1307; Boom et al. (2000) J. Virol. Methods 84:1-14 or Lantz et al. (1997) J. Microbiol. Meth. 28:159-167).

Potential PCR inhibitors found in stool specimens include heme, bilirubin, polysaccharides, bile salts and microorganisms other than the organism(s) of interest. The presence of PCR inhibitors determines, to a large extent, the sensitivity of the PCR-based method. In addition, the non-uniformity of stool samples in terms of physical matter, target organisms, and associated background fecal flora makes extraction of DNA from fecal specimens highly variable from specimen to specimen in terms of both the yield and the purity of the DNA. Thus, a major challenge in developing PCR-based detection methods that are suitable for fecal specimens is the development of a suitable sample preparation step to overcome problems caused by PCR inhibitors and to improve the efficiency DNA isolation.

In existing procedures to isolate nucleic acids (DNA, RNA) from a fecal specimen, the sample preparation step typically involves the addition of a relatively large volume of a lysis buffer to fecal material in order to make the nucleic acids accessible for isolation. For example, Rasool et al. report a nucleic acid isolation procedure for PCR detection of gastroenteritis viruses in fecal specimens wherein a 10% suspension of fecal material is lysed in 9 volumes of lysis buffer (J. Virol. Methods 100 (2002), 1-16). The same article describes a fecal lysate obtained by incubating 100 μl of a 10% fecal suspension with 25 μl of lysis buffer. It is assumed that the lysate contains an equivalent of 10 mg of fecal sample and represents therefore an 8% (wt/vol) fecal lysate. Boom et al. (J. Virol. Methods 84 (2000) 1-4) describe the detection and quantification of human cytomegalovirus DNA in feces wherein fecal material (a 25-50% [vol/vol] suspension) is lysed in 20 volumes of lysis buffer. In the method of Beld et al. to detect and quantify Hepatitis C virus RNA in feces, 900 μl lysis buffer is added to 50 μl of a 30% [vol/vol] fecal suspension (J Clinical Microbiology 2000, Vol. 38, No. 9 p. 3442-3444). Van der Hoek et al. (J. of Clinical Microbiology 1995, Vol. 33, No. 3 p. 581-588) report two ways to prepare a lysate of a fecal specimen: (i) feces is mixed with broth (30% [vol/vol]) and subsequently 100 μl of the suspension was added to 1200 μl of lysis buffer resulting in a 2.5% [vol/vol] fecal lysate, or (ii) approximately 50 mg of feces was added directly to 1200 μl of lysis buffer to yield a 4.16% (wt/vol) fecal lysate. Chui et al. (Diagnostic Microbiology and Infectious Disease, Vol. 48, no. 1, pp. 39-45) disclose the preparation of 10% (wt/vol) fecal lysate used in a PCR experiment for the detection of Mycobacterium avium. Brian et al. (J. Clin. Microbiol. 1992, p. 1801-1806) disclose a method for preparing a heat-lysate of stool consisting in suspending 100 mg of sample in 0.5 ml of buffer thereby obtaining a 20% (wt/vol) fecal lysate. The lysate is then further purified and PCR is performed to detect the presence of E. coli in the stool sample. PCT International Publication WO 92/00983, the contents of which are incorporated by this reference, describes the 4-fold dilution of a fecal sample (diluted in transport medium to an unspecified concentration) in a lysis solution prior to nucleic acid extraction in a method to determine the presence of Bacteroides gingivalis in the sample.

### DISCLOSURE OF THE INVENTION

The invention now provides the insight that significantly better results are obtained with PCR-technology applied to detect a microorganism in a fecal specimen if (e.g., during the sample preparation step) feces is mixed with a smaller relative volume of lysis buffer compared to the relative volumes of lysis buffer that have been used thus far.

Provided is a method for detecting the presence of a microorganism in a fecal specimen, characterized in that the method comprises the preparation of a 25-50% (wt/vol) fecal lysate.

In a preferred embodiment of a method of the invention, a 35-50% (wt/vol) fecal lysate, more preferred a 40-50% (wt/vol) lysate is prepared. For example, 100 mg feces is mixed with 200 μl of a lysis buffer to yield a 50% (wt/vol) fecal lysate, or a 50 mg feces sample is lysed in 125 μl lysis buffer resulting in a 40% (wt/vol) fecal lysate. The term “feces” as used herein generally refers to an undiluted fecal specimen obtained from a subject for example, a stool sample. A fecal sample may be of human or animal origin.

A fecal lysate according to the invention is typically prepared by mixing feces (e.g., a stool specimen) with a liquid capable of extracting DNA from the fecal material, such as a lysis buffer. Following mixing and centrifugation of the fecal suspension, a supernatant is obtained comprising among others nucleic acid material extracted from the fecal material. In a preferred embodiment of the invention, a method further comprises the steps of b) isolating a nucleic acid from the fecal lysate; c) subjecting the nucleic acid to a nucleic acid amplification assay using a set of at least two nucleic acid amplification primers specific for the microorganism of interest; and d) detecting amplified nucleic acid to determine whether the microorganism is present in the fecal specimen. A lysis buffer for use in a method according to the invention preferably comprises a chaotropic salt. This allows to subject the supernatant obtained from the fecal lysate in the sample preparation step (herein referred to as step a) to a highly efficient DNA isolation method reported by Boom et al. (Boom et al., (1990 “Rapid and simple method for purification of nucleic acids”; J. Clin. Microbiol. 28:495-503). This method is essentially based on the binding of nucleic acids to an acid-treated silica matrix in the presence of a chaotropic salt. According to this published method, the DNA is then eluted from the silica matrix using a low-salt solution.

In step c of a method according to the invention to detect a microorganism of interest in a fecal specimen, a nucleic acid isolated from a fecal lysate is subjected to a nucleic acid amplification assay using a set of at least two nucleic acid amplification primers specific for the microorganism of interest. In a preferred embodiment, the nucleic acid amplification assay comprises a polymerase chain reaction (PCR) technology, preferably Real Time (RT)-PCR.

A method according to the invention is advantageously used to quickly detect, quantify and identify a microorganism of interest in a fecal sample. Microorganisms which can be detected essentially include all microorganisms, such as viruses, bacteria and protozoa, which may be present in a fecal specimen. Of course, it is preferred that specific nucleic acid probes are available or can be designed to detect the microorganism using a nucleic acid amplification assay. In a preferred embodiment, the microorganism is a bacterium such as a pathogenic bacterium, preferably selected from the group essentially consisting of Salmonella species, Campylobacter species, Shigella species and Escherichia species such as enteropathogenic Escherichia coli, Salmonella enterica typhimurium, and Shigella flexneri.

With a method of the invention, it is possible to detect a microorganism, for example a pathogenic bacterium such as a Salmonella species with high sensitivity and high specificity. It allows to detect Salmonella enterica strains from the epidemiologically important serotypes including Agona, Bovismofibicans, Barncaster, Bradenburg, Braenderup, Bredeney, Broughton, Cannstatt, Cremieu, Deby, Dublin, Enetritidis, Eppendorf, Falkensee, Hadar, Heidelberg, Indiana, Infantis, Kottbus, Krefeld, Livingstone, Mbandaka, Montevideo, Niloese, Putten, Saint Paul, Senftenberg, Taksony, Tennessee, Thomson, Typhimurium, Virchow and 4.12:b:-.

In a specific embodiment of the invention, a nucleic acid extract is obtained from a fecal lysate according to the invention and this extract is subsequently subjected to a PCR assay to detect the presence of a Salmonella spp. in a fecal specimen using at least one nucleic acid amplification primer that is selected from the nucleic acid sequences set out in Table I.

The results obtained with a method according to the invention were compared with those obtained using three commonly used commercial DNA-isolation kits. Both the recovery of DNA isolated from feces was determined (see, Example 1), as well as the performance of the isolated DNA in a Real-Time PCR assay to detect Salmonella invA (see, Example 2). The average recovery of DNA when using a method as provided herein was 86%, versus an average recovery of 50-82% achieved with commercial kits. The percentage of samples with a recovery of 50% or more was 96% when using a method as provided herein, versus 44-87% for the commercial kits. The novel DNA isolation procedure also showed very good results with respect to the quality of the isolated and the suitability of the DNA extract for PCR analysis. Although the PCR performance of a DNA extract according to the invention was comparable to that of a DNA extract obtained using one of the commercial kits, the DNA recovery when using such a kit was much lower compared to the method of the invention. Thus, in contrast to known extraction methods, a method as provided herein combines an optimal DNA recovery with a very good DNA quality (e.g., absence of PCR inhibitors). These improved results are surprising, since one would expect more co-extraction of PCR inhibitors when using a nucleic acid extract that is obtained from a more concentrated fecal lysate. The invention also provides the use of a method according to the invention to detect the presence of a microorganism in a fecal specimen.

In a further embodiment, the invention provides an oligonucleotide with a nucleic acid sequence shown in Table 2, consisting of 5′-CG TCA TCC CAT TAC CTA CC-3′ (SalmInvAF2) (SEQ ID NO:_), 5′-GAA CGT TGA AAA ACT GAG GA-3′ (SalmInvAR2) (SEQ ID NO:_), 5′G AAA TGT TGA AAA GCT AAG GA-3′ (SalmInvAR3) (SEQ ID NO:_), 5′-TCT GGT TGA TTT TCT GAT CGC A-3′ (SalmInvAP2) (SEQ ID NO:_), 5′-CT GGT TGA TTT TCT GAT CGC G-3′ (SalmInvAP3) (SEQ ID NO:_), and 5′-CT GGT TGA TTT CCT GAT CGC G-3′ (SalmlnvAP4) (SEQ ID NO:_). The oligonucleotide is advantageously used to detect a Salmonella spp. nucleic amplification primer or a nucleic acid target probe (also known as detection probe). In a preferred embodiment, a nucleic acid amplification primer is selected from the group consisting of SalmInvAF2, SalmInvAR2 and SalmInvAR3 and/or a nucleic acid target probe is selected from the group consisting of SalmInvAP2, SalmInvAP3 and SalmInvAP4.

It will be apparent to a person skilled in the art that variant primers and probes other than those of Table 2 may be designed and used for amplification and detection of Salmonella spp. For instance, it is possible to use somewhat shorter or somewhat longer nucleotides, or oligonucleotides containing, for example, inositol residues or ambiguous bases or even oligonucleotides that contain one or more mismatches when compared to the sequences shown in Table 2. In general, variant oligonucleotides (which are herein defined as sequences that exhibit at least 65%, more preferably at least 80% homology with the oligonucleotide sequences of Table 2) are considered suitable for use in a method of the present invention.

A probe is preferably conjugated with a dye, such as a fluorochrome, more preferred with two dyes to allow detection of the probe. For example, a nucleic acid target probe according to the invention is labeled at the 5′ end with a reporter fluorochrome (e.g., 6-carboxyfluorescein, 6-FAM) and a quencher fluorochrome (e.g., 6-carboxy-tetramethyl-rhodamine, TAMRA) added at any T position or at the 3′ end. Such a probe is a so-called Taqman probe which can be used in Real Time (RT)-PCR assays using a Taq-polymerase. As long as both fluorochromes are on the probe, the quencher molecule stops all fluorescence by the reporter. However, as Taq polymerase extends the primer, the intrinsic nuclease activity of Taq-polymerase degrades the probe, releasing the reporter fluorochrome. Thus, the amount of fluorescence released during the amplification cycle is proportional to the amount of product generated in each cycle. For more information regarding RT-PCR and the Taqman technology see for example http://www.genetics.pitt.edu/taqman/.

Use of an amplification primer and/or a target probe according to the invention is provided to detect a Salmonella species in a sample, preferably using (RT)-PCR-based technology. A sample can be any type of biological sample or specimen, including patient stools or vomits, a water sample or a food sample. In one embodiment, an amplification primer and/or a target probe according to the invention is used to detect a Salmonella species in veterinary applications. The sample may or may not be enriched for Salmonella. Following sample preparation and DNA extraction, Salmonella DNA is amplified in a nucleic acid amplification assay to detect a Salmonella species using at least one primer selected from the primers set out in Table II.

The amplified DNA can be detected using a target probe of the invention. Of course, when detecting a Salmonella spp. in a fecal specimen it is preferred for the speed and sensitivity of detection to extract DNA from a fecal lysate using a method according to the present invention.

A Salmonella assay as provided herein appears to be highly suitable for large scale analyses, for example, in a clinical diagnostic setting, of clinical samples. A total number of 215 fecal samples that were positive in a conventional Salmonella-culturing diagnostic test, 196 samples tested positive and 19 samples tested negative in a PCR-assay of the invention (performed essentially as described in Example 1). This gives a sensitivity of 91.2%. When a total number of 152 fecal samples that were negative in a conventional Salmonella-culturing diagnostic test, 151 samples were tested negative and 1 sample tested positive in an assay of the invention. This indicates a specificity of more than 99%. The good test results of such a large number of clinical samples clearly illustrates that an assay as provided herein is advantageously used in a routine diagnostics, such as in a clinical setting.

In a further embodiment, the invention provides a kit for the detection of a Salmonella species, comprising a pair of nucleic acid amplification primers specific for a Salmonella species, wherein at least one primer or probe is selected from the nucleic acid sequences set out in Table 2, or a variant thereof.

The invention is further described with the aid of the following illustrative Examples.

### EXAMPLES

### Example 1

Detection of Salmonella Species in a Fecal Sample Using Real-Time-PCR (RT-PCR)

Sample Pretreatment

Feces was stored until use at −20° C. The sample was pretreated for DNA extraction as follows. A 35-50% (wt/vol) fecal lysate was prepared in lysis buffer L6 (5.25 M GuSCN (guanidinium thiocyanate), 50 mM Tris-HCl [pH 6.4], 20 mM EDTA (ethylenediamine tetra acetic acid), 1.3% (wt/vol) Triton X-100). For example, 100 mg feces was mixed with 200 μl lysis buffer L6 to yield a 50% (wt/vol) fecal suspension, or 50 mg feces was mixed with 125 μl L6 buffer to give a 40% (wt/vol) fecal suspension.

The lysate was vortexed and subsequently shaken for 20 minutes using a shaking apparatus such as a mini-beadbeater-8 at a low number of revolutions to ensure gentle shaking of the suspension. Thereafter, the lysate was centrifuged for 2 minutes in a bench top centrifuge at 12000×g and 100 μl supernatant was used as input in the subsequent DNA isolation step.

DNA Isolation

DNA isolation was performed essentially according to the method of Boom et al. The protocol for DNA extraction used in the present invention is based on publications by Boom et al. (1990) “Rapid and simple method for purification of nucleic acids”; J. Clin. Microbiol. 28:495-503; by Boom et al (2000) “Detection and quantitation of human cytomegalovirus DNA in feces”; J. Virol. Methods. 84:1-14) and by Beld et al. (2000) “Detection and quantitation of hepatitis C virus RNA in feces of chronically infected individuals” 2000. J. Clin. Microbiol. 38:3442-4.). In short, the following procedure was used.

Procedure:

900 μl L6 (5.25 M GuSCN, 50 mM Tris-HCl [pH 6.4], 20 mM EDTA, 1.3% (wt/vol) Triton X-100) was added to 50 μl SC-F (size-fractionated silicon dioxide) for feces prepared as described by Beld et al.

To the suspension of silica-particles, 100 μl of the nucleic acid-containing fecal-supernatant (obtained in the sample pretreatment step described above) was added. This mixture was vortexed and subsequently incubated for 10 minutes at room temperature. Following incubation, the mixture was vortexed again and centrifuged for 15 seconds in a bench top centrifuge at 12,000×g.

The resulting supernatant was removed and the silica-nucleic acid was washed twice with L2 (5.25 M GuSCN, 50 mM Tris-HCl [pH 6.4]), twice with 70% (vol/vol) ethanol and once with acetone. Following the last washing step with acetone, the silica-nucleic acid pellet was dried at 56° C. in a heating block. To elute the nucleic acids from the silica particles, 100 μl TE buffer (10 mM Tris-HCl, 1 mM EDTA, pH 8,0) was added followed by vortexing and a 10-minutes incubation at 56° C. The mixture was centrifuged for 2 minutes in a bench top centrifuge at 12,000×g. The resulting supernatant was used for the subsequent PCR analysis.

DNA Amplification and Detection of Amplified Products by RT-PCR

Real-Time PCR was performed using an ABI Prism 7700 Sequence Detection System using a PCR mix (25 μl) comprising the following components; 1× TaqMan Universal PCR Master Mix, 150 nM SalmInvAF1 (Styinva-JHO-2-left), 150 nM SalmInvAF2, 75 nM SalmInvAR1 (Styinva-JHO-2-right), 75 nM SalmInvAR2, 150 nM SalmInvAR3, 100 nM SalmInvAP1 (target probe), 100 nM SalmInvAP2, 100 nM SalmInvAP3, 100 nM SalmInvAP4, 0.5 μg BSA (Roche, 711 454) and 5 μl DNA eluate (the PCR mix was adjusted to a total volume of 25 μl with “water for molecular biology” (Sigma, W 4502)). Table 1 shows the sequences of the nucleic acid amplification primers and the target probes that were used. Table 2 only contains the novel oligonucleotide sequences from Table 1 that can be used either as primer or probe.

The mix was analyzed in the ABI Prism 7700 SDS system using the following thermoprofile; 2 minutes at 50° C., 10 minutes at 95° C., followed by 45 cycles at 15 seconds at 95° C., 15 seconds at 50° C. and 60 seconds at 60° C. The results obtained were analyzed using the sequence detection software from Applied Biosystems. From each sample to be analyzed, a PCR reaction was performed in duplicate; one without a spike and one with a spike of 15 pg Salmonella virchow DNA, to monitor the inhibitory effect on the PCR reaction.

TABLE 1

| | | |
|---|---|---|
| Name | Sequence | Reference |

| | | |
|---|---|---|
| SalmInvAF1 | 5′-TCG TCA TTC CAT TAC CTA CC-3′ | Hoorfar et al. |
| (Styinva-JHO-2-left) | (SEQ ID NO:_) | |
| SalmInvAF2 | 5′-CG TCA TCC CAT TAC CTA CC-3′ | present invention |
| | (SEQ ID NO:_) | |
| SalmInvAR1 | 5′-AAA CGT TGA AAA ACT GAG GA-3′ | Hoorfar et al. |
| (Styinva-JHO-2-right) | (SEQ ID NO:_) | |
| SalmInvAR2 | 5′-GAA CGT TGA AAA ACT GAG GA-3′ | present invention |
| | (SEQ ID NO:_) | |
| SalmInvAR3 | 5′-G AAA TGT TGA AAA GCT AAG GA-3′ | present invention |
| | (SEQ ID NO:_) | |
| SalmInvAP1 | 5′-TCT GGT TGA TTT CCT GAT CGC A-3′ | Hoorfar et al. |
| (target probe) | (SEQ ID NO:_) | |
| SalmInvAP2 | 5′-TCT GGT TGA TTT TCT GAT CGC A-3′ | present invention |
| | (SEQ ID NO:_) | |
| SalmInvAP3 | 5′-CT GGT TGA TTT TCT GAT CGC G-3′ | present invention |
| | (SEQ ID NO:_) | |
| SalmInvAP4 | 5′-CT GGT TGA TTT CCT GAT CGC G-3′ | present invention |
| | (SEQ ID NO:_) | |

Horrfar et al: Hoorfar, J., P. Ahrens, and P. R\dot{a}dström. Automated 5′ nuclease PCR assay for identification of Salmonella enterica. 2000. J. Clin. Microbiol. 38:3429-3435.

TABLE 2

| | |
|---|---|
| Name | Sequence |

| | |
|---|---|
| SalmInvAF2 | 5′-CG TCA TCC CAT TAC CTA CC-3′ |
| | (SEQ ID NO:_) |
| SalmInvAR2 | 5′-GAA CGT TGA AAA ACT GAG GA-3′ |
| | (SEQ ID NO:_) |
| SalmInvAR3 | 5′-G AAA TGT TGA AAA GCT AAG GA-3′ |
| | (SEQ ID NO:_) |
| SalmInvAP2 | 5′-TCT GGT TGA TTT TCT GAT CGC A-3′ |
| | (SEQ ID NO:_) |
| SalmInvAP3 | 5′-CG GGT TGA TTT TCT GAT CGC G-3′ |
| | (SEQ ID NO:_) |
| SalmInvAP4 | 5′-CT GGT TGA TTT CCT GAT CGC G-3′ |
| | (SEQ ID NO:_) |

### Example 2

### Performance of the Novel DNA Extraction Method

In this example, the performance of the novel method for extracting DNA from feces was compared with the procedures of three widely used commercial DNA extraction kits. Subsequently, the DNA extracts were subjected to a PCR analysis for the detection of a micro-organism (in this case Salmonella spp.)

Ideally, a DNA isolation procedure to obtain a fecal DNA extract should meet the following criteria:
1. DNA recovery is high, preferably 100%, although for feces a minimal recovery of 50% is acceptable.
2. The DNA extract obtained should contain no factors which can disturb subsequent analytical steps (e.g., polymerase inhibitors).

The comparison was made on the basis of the following criteria:
a) a good DNA recovery for an arbitrary sample was judged as a recovery of ≧50%.
b) A bad recovery for an arbitrary sample was judged as a recovery of <50%.
c) The average recovery of all samples within a method should be as high as possible.
d) No inhibition should be observed in the PCR assay.
e) In clinical samples, the agreement with the gold standard (culturing for the micro-organism) should be maximal.
   Material and Methods

Methods
1. Method of the invention, herein further referred to as “Boom SLGD”
2. Roche High Pure PCR Template Kit (HPPT)
3. Roche MagNA Pure LC DNA Isolation Kit III (Bacteria, Fungi) (automated extraction) (MPLC)
4. QIAGEN Stool DNA Mini Kit
   Recovery Experiments

Recovery analysis BOOM SLGD: 100 fecal samples that were randomly selected from routine diagnostic samples for fecal analysis (1 Salmonella culture positive-sample). DNA was extracted as described in Example 1.

Recovery analysis HPPT, MPLC, QIAGEN: 46 samples which were positive in a Salmonella culture test and 32 Salmonella negative fecal samples. DNA was extracted according to the manufacturer's instructions.

In all four different DNA extraction methods, the sample to be extracted was spiked with 4 μg HindIII digested phage λ DNA fragments.

Following DNA isolation, the amount of DNA present in the DNA extract was compared to a 100% recovery marker using agarose gel electrophoresis. The recovery was visually estimated by 4 individuals to yield a mean percentage of recovery for a given sample. To determine the total recovery performance of each of the 4 methods tested, the mean recoveries of all samples extracted with a certain method were averaged.

Recovery Results

The average DNA recovery of the samples (kits n=78, Boom SLGD n=100) and the percentages of these samples which showed a good (≧50%) or bad (<50%) recovery are shown in Table 3.

TABLE 3

| | | | | Boom- |
|---|---|---|---|---|
| | Roche MPLC | Roche HPPT | QIAGEN | SLGD |
| average recovery | 54% | 50% | 82% | 86% |
| Percentage | 56% | 59% | 13% | 4% |
| recovery <50% | | | | |
| Percentage | 44% | 41% | 87% | 96% |
| recovery ≧50% | | | | |

Roche MPLC = Roche MagNA Pure LC DNA III Kit Bacteria
Roche HPPT = Roche High Pure PCR Template Kit
QIAGEN = QIAGEN Stool DNA Mini Kit

Real-Time PCR Analysis

All DNA extracts obtained as described above were subjected to a Salmonella invA Real-Time PCR analysis using the procedure described in Example 1. In each case, the extract was analyzed in the absence or presence of 15 pg S. virchow DNA as a spike.

Real-Time PCR Results

The results of the RT-PCR analysis of Salmonella culture-positive samples are shown in Table 4A, 4B and 4C. It was not possible to compare each sample for all four different extraction methods in parallel. Therefore, each of the three commercial methods was compared to the novel method of the invention (Boom SLGD). No PCR inhibition was observed as evidenced by optimal amplification of the spike DNA. Table 4A shows that of the 43 fecal specimens tested (all known to be Salmonella positive on the basis of culturing tests), 30 specimens tested positive when extracted with the Boom SLGD method as well as with the commercial Roche MPLC kit. Seven specimens tested negative using either one of the extraction methods, whereas 3 specimens tested negative with one method yet positive with the other. Tables 4A and 4B indicate that, with respect to the suitability for PCR analysis, an extract obtained with the Boom SLGD method is of approximately equal quality when compared to extracts prepared using the Roche MPLC or Roche HPPT kit. In contrast, the Boom SLGD method allows to detect more Salmonella positive specimens when compared to the QIAGEN kit; 35 (out of the 46) specimens were identified as positive when Boom SLGD extracts were prepared, versus 32 when the QIAGEN kit was used. It should be noted that the Boom SLGD procedure was tested as the last of the four procedures. This means that any detrimental effects of repeated freeze-thawing of the fecal samples (e.g., DNA degradation) were at the expense of the Boom SLGD procedure.

TABLE 4A
Roche MPLC kit compared to Boom SLGD

| | | Boom SLGD | | |
|---|---|---|---|---|
| | | + | − | total |
| Roche MPLC | + | 30 | 3 | 33 |
| | − | 3 | 7 | 10 |
| | total | 33 | 10 | 43 |

TABLE 4B
Roche HPPT kit compared to Boom SLGD

| | | Boom SLGD | | |
|---|---|---|---|---|
| | | + | − | total |
| Roche HPPT | + | 33 | 3 | 36 |
| | − | 2 | 8 | 10 |
| | total | 35 | 11 | 46 |

TABLE 4C
QIAGEN kit compared to Boom SLGD

| | | Boom SLGD | | |
|---|---|---|---|---|
| | | + | − | total |
| QIAGEN | + | 31 | 1 | 32 |
| | − | 4 | 10 | 14 |
| | total | 35 | 11 | 46 |

### CONCLUSION

The average DNA recovery when using a Boom SLGD method of the invention is 86%, as is shown in Table 3. This is higher than any of the commercial kits (54% en 50% for the Roche methods versus 82% for QIAGEN). In addition, the majority (96%) of the Boom SLGD samples shows a good or satisfactory recovery (recovery ≧50%). With the commercial kits, a satisfactory recovery was obtained in only 41-44% of the cases for Roche kits and 87% for QIAGEN.

From Tables 4A-4C it can be concluded that the performance in the PCR assay of the Boom SLGD method is comparable to the Roche MPLC and HPPT kits. The Boom SLGD performs better than the QIAGEN kit.

Taken together, the Boom SLGD performs very well with respect to the average DNA recovery as well as the quality of the DNA extract for subsequent PCR analysis. None of the commercial kits tested showed a good DNA recovery in combination with an optimal performance of the DNA in a PCR assay. Thus, a method provided by the present invention is advantageously used to detect a microorganism in a fecal sample using PCR technology.

What is claimed is:

1. An isolated nucleic acid sequence selected from the group of oligonucleofides consisting of 5′-CG TCA TCC CAT TAC CTA CC-3′ (SEQ ID NO:_); 5′-GAA CGT TGA AAA ACT GAG GA-3′ (SEQ ID NO:_); 5′-G AAA TGT TGA AAA GCT AAG GA-3′ (SEQ ID NO:_); 5′-TCT GGT TGA TTT TCT GAT CGC A-3′ (SEQ ID NO:_); 5′-CT GGT TGA TTT TCT GAT CGC G-3′ (SEQ ID NO:_); 5′-CT GGT TGA TTT CCT GAT CGC G-3′ (SEQ ID NO:_), and a variant of any thereof having at least 65% homology with the oligonucleotide.

2. A kit for detecting a Salmonella species, said kit comprising:
the isolated nucleic acid sequence of claim 1.

3. A nucleic acid amplification assay to detect a Salmonella species, said nucleic acid amplification assay comprising:
the isolated nucleic acid sequence of claim 1.

4. A kit for detecting a Salmonella species, said kit comprising:
a pair of nucleic acid amplification primers and a nucleic acid target probe wherein at least one nucleic acid amplification primer and/or nucleic acid target probe is selected from the group of oligonucleotides consisting of 5′-CG TCA TCC CAT TAC CTA CC-3′ (SEQ ID NO:_); 5′-GAA CGT TGA AAA ACT GAG GA-3′ (SEQ ID NO:_); 5′-G AAA TGT TGA AAA GCT AAG GA-3′ (SEQ ID NO:_); 5′-TCT GGT TGA TTT TCT GAT CGC A-3′ (SEQ ID NO:_); 5′-CT GGT TGA TTT TCT GAT CGC G-3′ (SEQ ID NO:_); 5′-CT GGT TGA TTT CCT GAT CGC G-3′ (SEQ ID NO:_), and a variant of any thereof having at least 65% homology with the oligonucleotide.

5. A method for detecting a microorganism of interest in a fecal specimen, wherein the method comprises:
a) preparing a 25-50% (wt/vol) fecal lysate from the specimen.

6. The method according to claim 5, further comprising:
b) isolating nucleic acid from said fecal lysate;
c) subjecting said nucleic acid to a nucleic acid amplification assay using a set of at least two nucleic acid amplification primers specific for the microorganism of interest;
d) detecting amplified nucleic acid to determine whether or not said microorganism is present in said fecal lysate.

7. The method according to claim 6, wherein said nucleic acid amplification assay comprises polymerase chain reaction (PCR) technology or Real Time-PCR.

8. The method according to claim 5, wherein the microorganism of interest is selected from the group consisting of a pathogenic bacterium, Salmonella species, Campylobacter species, Shigella species, Escherichia species, Staphylococcus species, Vibrio species, and Clostridium species.

9. The method according to claim 6, wherein the microorganism of interest is selected from the group consisting of a pathogenic bacterium, Salmonella species, Campylobacter species, Shigella species, Escherichia species, Staphylococcus species, Vibrio species, and Clostridium species.

10. The method according to claim 7, wherein the microorganism of interest is selected from the group consisting of a pathogenic bacterium, Salmonella species, Campylobacter species, Shigella species, Escherichia species, Staphylococcus species, Vibrio species, and Clostridium species.

11. The method according to claim 5, wherein a Salmonella species is detected using at least one nucleic acid amplification primer or target probe selected from the oligonucleotides set out in Table 1.

12. The method according to claim 5, wherein a Salmonella species is detected using at least one nucleic acid amplification primer or nucleic acid target probe, wherein the at least one nucleic acid amplification primer and/or nucleic acid target probe is selected from the group of oligonucleotides consisting of 5′-CG TCA TCC CAT TAC CTA CC-3′(SEQ ID NO:_); 5′-GAA CGT TGA AAA ACT GAG TGA-3′(SEQ ID NO:_); 5′-G AAA TGT TGA AAA GCT AAG GA-3′(SEQ ID NO:_); 5′-TCT GGT TGA TTT TCT GAT CGC A-3′(SEQ ID NO:_); 5′-CTGGT TGA TTT TCT GAT CGC G-3′ (SEQ ID NO:_); 5′-CT GGT TGA TTT CCT GAT CGC G-3′(SEQ ID NO:_), and a variant of any thereof having at least 65% homology with the oligonucleotide.

13. The method according to claim 5, wherein the fecal lysate is a 35-50% (wt/vol) fecal lysate.

14. The method according to claim 5, wherein the fecal lysate is a 40-50% (wt/vol) fecal lysate.


SEQUENCE LISTING (9 sequences)

SEQ ID NO: 1 - DNA, 20 bp, Artificial
  SalmInvAF1 - Primer/Probe
tcgtcattccattacctacc

SEQ ID NO: 2 - DNA, 19 bp, Artificial
  SalmInvAF2  - Primer/Probe
cgtcatcccattacctacc

SEQ ID NO: 3 - DNA, 20 bp, Artificial
  SalmInvAR1 - Primer/Probe
aaacgttgaaaaactgagga

SEQ ID NO: 4 - DNA, 20 bp, Artificial
  SalmInvAR2 - Primer/Probe
gaacgttgaaaaactgagga

SEQ ID NO: 5 - DNA, 21 bp, Artificial
  SalmInvAR3 - Primer/Probe
gaaatgttgaaaagctaagga

SEQ ID NO: 6 - DNA, 22 bp, Artificial
  SalmInvAP1 - Primer/Probe
tctggttgatttcctgatcgca

SEQ ID NO: 7 - DNA, 22 bp, Artificial
  SalmInvAP2 - Primer/Probe
tctggttgattttctgatcgca

SEQ ID NO: 8 - DNA, 21 bp, Artificial
  SalmInvAP3 - Primer/Probe
ctggttgattttctgatcgcg

SEQ ID NO: 9 - DNA, 21 bp, Artificial
  SalmInvAP4 - Primer/Probe
ctggttgatttcctgatcgcg